(19) United States
(12) Patent Application Publication
(10) Pub. No.: US 2011/0282201 A1
(43) Pub. Date: Nov. 17, 2011

(54) DEVICE CAPABLE OF TESTING BONE DENSITY AND SYSTEM THEREOF

(75) Inventor: CHIEN-FENG HUANG, Tu-Cheng (TW)

(73) Assignee: HON HAI PRECISION INDUSTRY CO., LTD., Tu-Cheng (TW)

(21) Appl. No.: 12/862,702

(22) Filed: Aug. 24, 2010

(30) Foreign Application Priority Data
May 17, 2010 (TW) 99115611

(51) Int. Cl. A61B 8/00

(52) U.S. Cl. 600/438

(57) ABSTRACT

A system includes a testing device and a portable device. The testing device includes a first connector, a signal converting unit, an emitting member, and a receiving member. The portable device includes a second connector and a processor. The signal converting unit converts the test direct current through the first connector and the second connector to the pulse electrical signal. The emitting member converting the pulse electrical signal to an ultrasonic signal. The receiving member converts the ultrasonic signal to the electronic signal. The signal converting unit converts the electronic signal to the feedback digital signal, and transmits the feedback digital signal to the portable device through the first connector and the second connector. The processor includes a testing module and an analyzing module. The testing module calculates the interval to calculate speed. The analyzing module generates a data of the bone density according to the speed.

### BACKGROUND

1. Technical Field

The present disclosure relates to testing devices and systems thereof and, particularly, to a device capable of testing bone density and a system thereof.

2. Description of Related Art

Conventional bone density testing measurements require expensive equipment that is usually available only in specialized facilities. This kind of equipment is typically complex, and the test results must be interpreted by a certified person (e.g., a radiographer), resulting in a number of additional drawbacks: the cost of labor associated with the radiographers is significant; the test results must be interpreted by humans, increasing the possibility of human error. More specifically, because subjective judgment is involved, two radiographers may reach different conclusions as to the actual bone density when interpreting identical data. This is particularly problematic when measuring the bone density of a single patient over a period of time, such as at one year intervals, to determine the progression of the density of bone loss. It would be advantageous to remove these human variations, to determine more accurately the progression of osteoporosis over time.

The need for the interpretation of test results also makes it more difficult to return these results to the patient instantly. It would be advantageous to measure bone density as easily as taking a blood pressure reading.

### BRIEF DESCRIPTION OF THE DRAWINGS

The components of the drawings are not necessarily drawn to scale, the emphasis instead being placed upon clearly illustrating the principles of a device capable of testing bone density and a system thereof. Moreover, in the drawings, like reference numerals designate corresponding parts throughout several views.

FIG. 1 is a schematic view of a testing device in accordance with an exemplary embodiment.

FIG. 2 is a schematic view of a system in accordance with an exemplary embodiment, showing the testing device and a portable device.

FIG. 3 is similar to FIG. 2, showing a second embodiment of the testing device and the portable device.

FIG. 4 is a block diagram of the system of FIG. 2.

### DETAILED DESCRIPTION

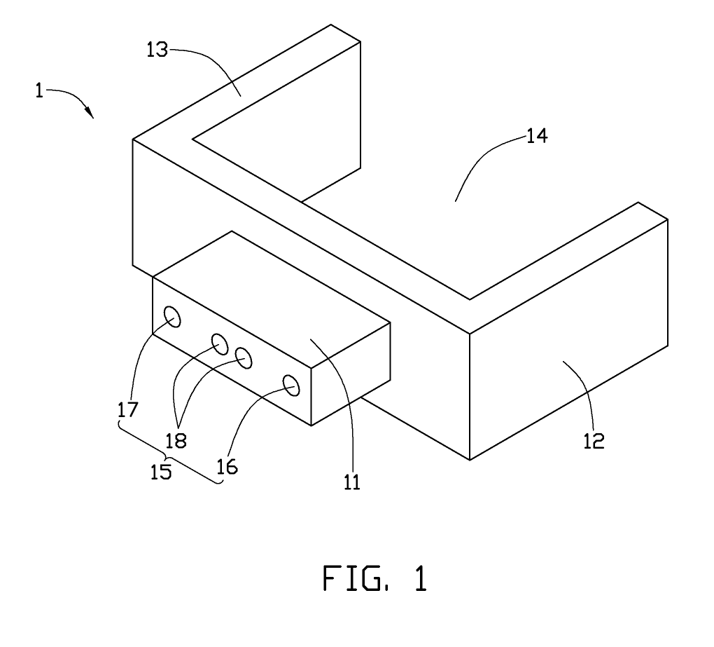

Referring to FIG. 1, a testing device 1 in accordance with an exemplary embodiment is shown. The testing device 1 is for use with a portable device to test bone density. In the embodiment, the portable device could be a modified mobile phone for mobility.

The testing device 1 includes a main body 11, an emitting member 12 and a receiving member 13. The emitting member 12 and the receiving member 13 protrude from opposite ends of the main body 11. The main body 11, the emitting member 12 and the receiving member 13 define a receiving space 14. The emitting member 12 and the receiving member 13 are transducers made of piezoelectric material, for example quartz crystal. The emitting member 12 converts a received high frequency electrical signal to an oscillation to generate an ultrasonic signal. The receiving member 13 receives the ultrasonic signal, and converts the ultrasonic signal to an electrical signal.

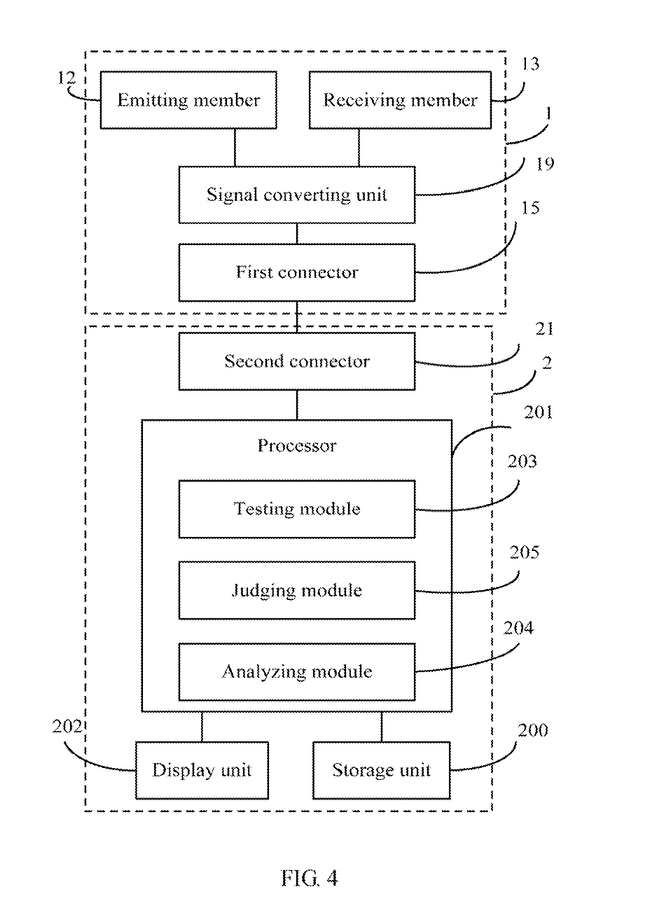

The main body 11 further includes a first connector 15 for connecting the testing device 1 to the portable device. The first connector 15 includes an emitting pin 16, a receiving pin 17 and a power pin 18. The testing device 1 receives power from the portable device through the power pin 18. The testing device 1 further includes two signal converting units 19 (see FIG. 4). In the embodiment, one signal converting unit 19 is arranged between the main body 11 and the emitting member 12. The other signal converting unit 19 is arranged between the main body 11 and the receiving member 13.

When in use, one hand of a patient is placed in the receiving space 14. The signal converting unit 19 converts the testing DC (direct current) received from the portable device through the emitting pin 16 into a pulse electrical signal, and transmits the pulse electrical signal to the emitting member 12. The emitting member 12 converts the pulse electrical signal into an ultrasonic signal. The ultrasonic signal passes through the hand and received by the receiving member 13. The receiving member 13 receives the ultrasonic signal and converts the ultrasonic signal into an electrical signal. The signal converting unit 19 converts the electrical signal to a feedback digital signal, and transmits the feedback digital signal to the portable device through the receiving pin 17 to analyze a general condition of the user's bone.

In the embodiment, the portable device can load a testing software. After the testing device 1 is connected to the portable device, the testing software can be activated to test bone density.

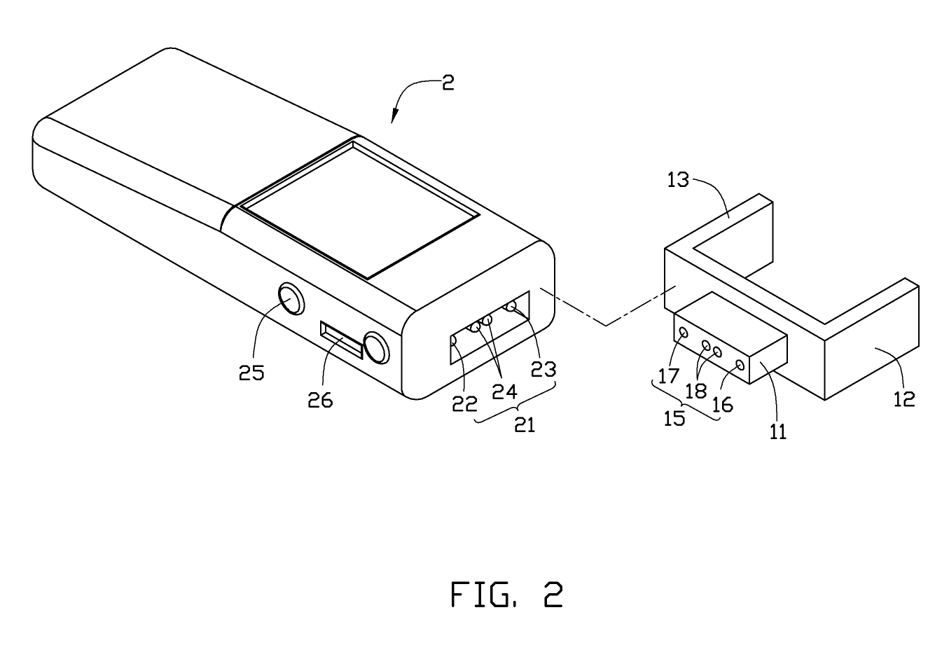

Referring to FIG. 2, a system in accordance with an exemplary embodiment is shown. The system includes a testing device 1 and a portable device 2.

The portable device 2 includes a second connector 21 corresponding to the first connector 15 of the testing device 1. The second connector 21 includes an emitting contact 22, a receiving contact 23 and a power contact 24. In the embodiment, when the testing device 1 is connected to the portable device 2, the emitting pin 16 contacts the emitting contact 22, the receiving pin 17 contacts the receiving contact 23, and the power pin 18 contacts the power contact 24.

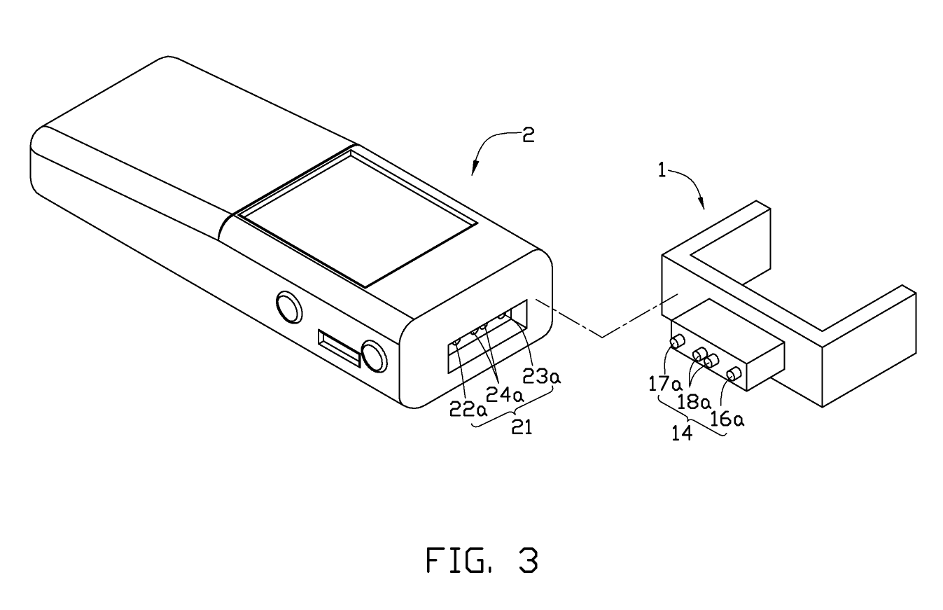

In an alternative embodiment as shown in FIG. 3, the first testing port 15 of the testing device 1 includes an emitting contact 16a, a receiving contact 17a, and a power contact 18a. The second testing port 21 of the portable device 2 includes an emitting pin 22a, a receiving pin 23a, and a power pin 24a.

Referring to FIG. 4, the portable device 2 further includes a storage unit 200, a processor 201, and a display unit 202. The storage unit 200 stores a table (not shown) listing of the bone density of different speeds of the ultrasonic transmissions. The processor 201 includes a testing module 203 and an analyzing module 204. When the ultrasonic signal passes through the different materials, the ultrasonic signal will transmit at different speeds. Different material corresponds to a different density. The testing module 203 is configured to calculate the interval between the time of the DC transmitted from the portable device 2 to the testing device 1 and that of the feedback digital signal received from the testing device 1, and further calculate the speed of the ultrasonic signals transmitting through the hand based on the calculated interval. The analyzing module 204 is configured to compare the speed of the ultrasonic signals passing through the hand and the speed of the table to generate data of the bone density. In an alternative embodiment, the analyzing module 204 is further configured to generate an advice about the patient's health based on the data of the bone density.

In the embodiment, the portable device 2 further includes a button 25 and a speaker 26. The button 25 and the speaker 26 are arranged on the sidewall of the portable device 2. The button 25 is configured to activate the testing module 202. The speaker 26 is configured to generate a sound after the operation of testing bone density to prompt that the test has been completed.

The processor 201 may further include a judging module 205. The judging module 205 is configured to determine whether an incoming call is received. At the beginning of the bone density test, if the judging module 205 determines an incoming call is received, the display unit 202 displays that an incoming call is being received. Otherwise the test can be performed, and the analyzing module 204 transmits the testing DC from the portable device 2 to the signal converting unit 19 of the testing device 1 through the emitting pin 16 and the emitting contact 22. The signal converting unit 19 converts the testing DC to a pulse electrical signal, and transmits the pulse electrical signal to the emitting member 12. The emitting member 12 converts the pulse electrical signal into the ultrasonic signal. The receiving member 13 receives the ultrasonic signal and converts the ultrasonic signal into the electrical signal. The signal converting unit 19 converts the electrical signal into the feedback digital signal, and transmits the feedback digital signal to the portable device 2 through the receiving pin 17 and the receiving contact 23. During the testing of the bone density, if the judging module 205 determines an incoming call is received, the analyzing module 204 interrupts the test and the display unit 202 displays that an incoming call is being received.

To test the bone density, the first connector 14 is connected to the second connector 21. The hand is placed in the receiving space 14 and the button 25 is pressed to start the bone density test. The judging module 205 determines whether an incoming call is received. If so, the display unit 202 displays that an incoming call is received and will not perform the test. If not, the portable device 2 executes the operation of bone density testing and displays the data on the display unit 202. During the test, the judging module 205 continuously determines whether an incoming call is received. If so, the analyzing module 204 interrupts the operation of the bone density testing and displays that an incoming call is received on the display unit 202. If not, the analyzing module 204 continues testing bone density, and closes the testing device 1 after the testing. After the testing, the speaker 26 generates a sound to prompt that the testing has completed.

Although the present disclosure has been specifically described on the basis of the exemplary embodiment thereof, the disclosure is not to be construed as being limited thereto. Various changes or modifications may be made to the embodiment without departing from the scope and spirit of the disclosure.

What is claimed is:

1. A testing device for use with a portable device to test bone density, the testing device comprising:
a connector configured to connect the testing device to the portable device;
a signal converting unit configured for converting a test direct current received from the portable device to a pulse electrical signal;
an emitting member configured for receiving the pulse electrical signal and converting the pulse electrical signal into an ultrasonic signal;
a receiving member configured for receiving the ultrasonic signal passing through a user's hand between the emitting member and the receiving member, and converting the ultrasonic signal into an electronic signal; and
the signal converting unit converting the electronic signal from the receiving member into a feedback digital signal, and transmitting the feedback digital signal to the portable device through the connector.

2. The testing device as described in claim 1, wherein the connector comprises an emitting pin, a receiving pin, and a power pin, the signal converting unit the testing device obtaining power from the portable device through the power pin and transmitting the feedback digital signal to the portable device through the receiving pin.

3. The testing device as described in claim 1, wherein the connector comprises an emitting contact, a receiving contact and a power contact, the testing device obtaining power from the portable device through the power contact, the signal converting unit receiving the test direct current from the portable device through the emitting contact, and transmitting the feedback digital signal to the portable device through the receiving contact.

4. The testing device as described in claim 1 further comprising a main body, the emitting member and the receiving member protrude from the main body, the main body, the emitting member and the receiving member defines a receiving space to receive the hand, the connector is defined in the main body.

5. The testing device as described in claim 1, wherein the emitting member and the receiving member are transducers made of piezoelectric material.

6. A system, comprising:
a testing device, comprising,
a first connector;
a signal converting unit converting the test direct current received from the portable device to the pulse electrical signal;
an emitting member receiving the pulse electrical signal and converting the pulse electrical signal to an ultrasonic signal;
a receiving member receiving the ultrasonic signal passing through one user's hand between the emitting member and the receiving member, and converting the ultrasonic signal to the electronic signal;
the signal converting unit converting the electronic signal to the feedback digital signal, and transmitting the feedback digital signal to the portable device; and
a portable device, comprising,
a second connector contacting the first connector to connect the testing device to the portable device, the signal converting unit receiving the test direct current from the portable device through the first connector and the second connector, and transmit the feedback digital signal to the portable device through the first connector and the second connector;
a storage unit storing a table listing each different speed and different density corresponding to each different speed;
a processor comprising a testing module and an analyzing module, the testing module calculating the interval between the time of the testing direct electrical signal transmitting from the portable device and the time of the feedback digital signal received by the portable device, and further calculating the speed of the ultrasonic transmitting through the hand, the analyzing module generating a data of the bone density according to the speed and the table stored in the storage unit; and
a display unit configured to display information.

7. The system as described in claim 6, wherein the portable device further comprises a button to activate the testing module to test the bone density in response to user operate.

8. The system as described in claim 6, wherein the first connector comprising an emitting pin, a receiving pin and a power pin, the second connector comprising an emitting contact, a receiving contact and a power contact, the testing device receives power from the portable device through the power pin and power contact, the signal converting unit receiving the test direct current from the portable device through the emitting pin and the emitting contact, and transmit the feedback digital signal to the portable device through the receiving pin and the receiving contact.

9. The system as described in claim 6, wherein the first connector comprises an emitting contact, a receiving contact, and a power contact, the second connector comprises an emitting pin, a receiving pin, and a power pin, the testing device receives power from the portable device through the power pin and power contact, the signal converting unit receiving the test direct current from the portable device through the emitting pin and the emitting contact, and transmit the feedback digital signal to the portable device through the receiving pin and the receiving contact.

10. The system as described in claim 6, wherein the emitting member and the receiving member are transducers made of piezoelectric material.

11. The system as described in claim 6 further comprising a main body, the emitting member and the receiving member protrude from the main body, the emitting member and the receiving member defines a receiving space to receive the hand, the first connector is defined in the main body.

12. The system as described in claim 6, wherein the processor further comprises a judging module, the judging module is configured to judge whether an incoming call is received at the beginning of the testing, when an incoming call is received, the display unit displays that an incoming call is received, when an incoming call is not received, the portable device tests bone density; during the test, when an incoming call is received, the analyzing module shutdowns the bone density testing and displays that an incoming call is received on the display unit, when an incoming call is not received, the analyzing module continues testing bone density, and closes the testing device after testing.
